(19) United States
(12) Patent Application Publication
(10) Pub. No.: US 2012/0095379 A1
(43) Pub. Date: Apr. 19, 2012

(54) PELVIC BELT

(75) Inventors: Seiji Hama, Hiroshima (JP); Masafumi Ootsubo, Hiroshima (JP); Setsuko Ootsubo, Hiroshima (JP)

(73) Assignee: HIROSHIMA UNIVERSITY, Higashi-Hiroshima-shi, Hiroshima (JP)

(21) Appl. No.: 12/994,924

(22) PCT Filed: May 11, 2009
(86) PCT No.: PCT/JP2009/002049

§ 371 Date: May 27, 2011

(30) Foreign Application Priority Data
May 28, 2008 (JP) 2008-140048

(51) Int. Cl. A61F 5/00

(52) U.S. Cl. 602/23

(57) ABSTRACT

A pelvic belt 1 includes a pressing member 2 positioned behind the hip joint of Patient A, and a pressing-member fixing band 3 wound around a portion corresponding to the pelvis 100 of Patient A. The pressing member 2 is fixed while being pushed toward the hip joint, thereby preventing the hip joint from becoming unstable and moving backward during walking, and making the walking posture more similar to a normal walking posture.

### TECHNICAL FIELD

The present invention relates to pelvic belts for use, e.g., for patients who have trouble walking due to such as paralysis.

### BACKGROUND ART

For example, patients who have strokes, neuromuscular diseases, etc., and patients who have spinal cord injuries may experience dysfunction of the lower limbs and the trunk due to such as paralysis. Further, the dysfunction may decrease the strength of muscles of the lower limbs and the trunk. When such a patient walks, the hip joint of the patient's lower limb in the stance phase becomes unstable and moves backward, causing the pelvis to sway and be unsettled. This makes the whole trunk of the patient sway unnaturally, and the body is considerably unbalanced. Thus, the patient has an abnormal walking posture, which is out of a range of normal walking postures, and thus has trouble walking.

Further, when a patient having a paralysis on one lower limb walks, the hip joint of the paralyzed lower limb in the stance phase becomes unstable and moves backward and the hips move backward. This causes hyperextention of the knee joint of the paralyzed lower limb. When the knee joint of the paralyzed lower limb is hyperextended, the patient cannot swing his/her healthy lower limb forward enough. Thus, the patient's walking posture is not efficient, and the patient has trouble walking.

Rehabilitation is provided for those patients who have trouble walking as described above to restore their functions. Various types of devices are invented for use in rehabilitation. For example, Patent Document 1 discloses a walking assistance device configured to provide an assisting force to the lower limbs during walking. This walking assistance device includes a hip support member affixed to the lower back of a patient, a lower limb support member affixed to the patient's lower limb, and a drive unit for generating an assisting force. The hip support member includes a back support which extends from portions corresponding to the left and right ilia to the backside, and a belt for fixing the back support to the patient's lower back. Further, the drive unit includes an electric motor equipped with a reduction gear, which is attached to connect the back support and the lower limb support member together.

### CITATION LIST

### Patent Document

PATENT DOCUMENT 1: Japanese Patent Publication No. 2005-000634

### SUMMARY OF THE INVENTION

### Technical Problem

However, according to the walking assistance device of the Patent Document 1, the back support, to which the drive unit is attached, needs to have high stiffness so that an effective assisting force can be applied to the lower limbs by the drive unit. In addition, the back support has to be firmly affixed to the lower back of the patient with the belt. If the back support is affixed in this manner, the pelvis of the patient is fixed to the back support, and can hardly move. Thus, pelvic movements in a normal range, such as a forward tilting and rotational movements, which also occurs in the non-disabled people are restricted. If the pelvic movements in a normal range are restricted, it is impossible for the patient to learn the normal movements of the pelvis. Thus, a facilitating effect on a muscle cannot be expected, and effective rehabilitation cannot be provided.

Further, the back support of the walking assistance device needs to be firmly affixed to the lower back of the patient. Thus, it is difficult to change the location at which the back support is affixed. This means that the location at which the device is affixed cannot be adjusted according to the paralytic condition and degree of restoration of the patient.

Moreover, the drive unit of the walking assistance device includes an electric motor equipped with a reduction gear, for which a control device and a power supply are necessary. This increases the cost of the device. As a result, it is difficult to make the devices widely available.

The present invention was made in view of the above problems, and it is an objective of the invention, when providing rehabilitation etc. to a patient having trouble walking, to have a sufficient facilitating effect on a muscle by not preventing a pelvic movement in a normal range, while making the walking posture of the patient more similar to a normal walking posture; to obtain a sufficient effect of treatment by making it possible to change the state of use according to the paralytic condition and degree of restoration of each patient; and further to achieve low cost.

### Solution to the Problem

To achieve the above objectives, it is possible in the present invention to fix a pressing member, which is for pressing the patient's hip joint from backside, to the patient with a fixing member.

Specifically, the first aspect of the present invention includes a pressing member for pressing a hip joint, and a fixing member for pushing the pressing member from backside toward the hip joint and fixing the pressing member.

According to this structure, the pressing member is positioned and fixed behind the hip joint, and thus, it possible to press the hip joint from backside by using the pressing member. This prevents the hip joint from becoming unstable and moving backward during walking, thereby reducing the sway of the pelvis. Thus, it is possible to make the walking posture of the patient more similar to a normal walking posture without applying an assisting force given by the drive unit as in the conventional case. Further, because such a drive unit is not necessary, the fixing member does not have to be fastened as firmly as the conventional belt, and may be tightened just enough to bring the pressing member pressed against the hip joint. Thus, the movement of the pelvis in a normal range is not interrupted by the pelvic belt, and it is possible to fine adjust the location at which the fixing member is wound, and the location of the pressing member, according to the paralytic condition and degree of restoration of each patient. Further, the pressing member is pressed against the hip joint by the fixing member, and thus, no control device or power supply is necessary. Accordingly, the structure can be simplified, compared to the case in which the drive unit is used.

The second aspect of the present invention is that in the first aspect of the present invention, the fixing member is configured to fix the pressing member to a dimple of the hips, the dimple extending from about an outer edge of the gluteus maximus along a horizontal direction toward a lateral side.

That is, in the human anatomy, no thick muscle like the gluteus maximus exists in an area of the hips that extends from about an outer edge of the gluteus maximus along a horizontal direction toward a lateral side. This means that a dimple is formed at the lateral side of the hips. The proximal end of the femur that forms part of the hip joint is located at a portion which corresponds to the inside of the dimple. Thus, the pressing force of the pressing member can be reliably applied to the hip joint by placing the pressing member in the dimple of the hip. Moreover, since placed in the dimple, the pressing member can be stabilized while the patient is walking.

The third aspect of the present invention is that in the first or second aspect of the present invention, a pressing surface of the pressing member is curved and protrudes.

According to this structure, the pressing member can be pushed against the hip joint without causing pain to the patient.

The fourth aspect of the present invention is that in any one of the first to third aspects of the present invention, the pressing member includes an insertion portion through which the fixing member is inserted.

According to this structure, the pressing member and the fixing member are linked to each other, with the fixing member inserted through the insertion portion of the pressing member.

The fifth aspect of the present invention is that in any one of the first to fourth aspects of the present invention, the fixing member has an elongated shape, and has an elastic portion that is elastic in a longitudinal direction of the fixing member.

According to this structure, it is possible to adjust the force pushing the pressing member to the hip joint, when the fixing member is wound around the patient, by changing the elasticity of the elastic portion.

The sixth aspect of the present invention includes, in any one of the first to fifth aspects of the present invention, a pelvis back support for covering a portion corresponding to the wing of ilium of the coxal bone and the sacrum.

According to this structure, the pelvis can be supported by the pelvis back support from backside.

The seventh aspect of the present invention includes, in any one of the first to sixth aspects of the present invention, a pubic support for covering a portion corresponding to the pubic symphysis of the coxal bone.

According to this structure, the pelvis can be supported by the pubic support from the front side.

### Advantages of the Invention

According to the first aspect of the present invention, the pressing member positioned at a portion corresponding to the backside of the hip joint can be pushed against the hip joint and fixed by the fixing member. Thus, it is possible to make the walking posture of the patient more similar to a normal walking posture without using the conventional drive unit. As a result, the movements of the pelvis in a normal range are not interrupted by the pelvic belt, thereby making it possible to have a sufficient facilitating effect on a muscle. Also, the location of the pressing member is fine adjusted according to the paralytic condition and degree of restoration of each patient, thereby making it possible to obtain a sufficient effect of treatment. Further, it is possible to simplify the structure and reduce costs.

According to the second aspect of the present invention, it is possible to avoid a thick muscle like the gluteus maximus, in placing the pressing member. It is also possible to reduce displacement of the pressing member. Thus, the pressing force of the pressing member can be reliably applied to the hip joint, and it is possible to effectively prevent the hip joint from becoming unstable and moving backward.

According to the third aspect of the present invention, the surface of the pressing member that is pushed toward the hip joint is curved. Thus, the pressing force can be reliably applied to the hip joint without causing pain to the patient when the pressing member is affixed to the patient.

According to the fourth aspect of the present invention, the pressing member and the fixing member are linked to each other, with the fixing member inserted through the insertion portion of the pressing member. This means that the location of the pressing member with respect to the fixing member can be easily changed, and thereby, the location to which the pressing force is applied can be adjusted.

According to the fifth aspect of the present invention, the fixing member has an elastic portion. Thus, the pressing force toward the hip joint can be easily adjusted.

According to the sixth aspect of the present invention, the patient's pelvis can be supported from backside. According to the seventh aspect of the present invention, the patient's pelvis can be supported from the front side. According to these aspects of the present invention, it is possible to make the walking posture of the patients with severe conditions, as well, more similar to a normal walking posture. Thus, the invention can be used for those patients with various conditions.

### BRIEF DESCRIPTION OF THE DRAWINGS

FIG. 1 shows the anatomy around the human pelvis and the hip joints, viewed from the backside.

FIG. 2 shows the anatomy around the human pelvis and the hip joints against which pressing members are pushed, viewed from above.

FIG. 3 shows a pelvic belt when used according to the first embodiment, viewed from the front side.

FIG. 4 shows a pelvic belt when used according to the first embodiment, viewed from the backside.

FIG. 5 shows a pelvic belt when used according to the first embodiment, viewed from the lateral side.

FIG. 6 shows an enlarged connecting portion for connecting a connecting band and a pubic support panel, according to a variation.

FIG. 7 is a drawing which corresponds to FIG. 3, according to the second embodiment.

FIG. 8 is a drawing which corresponds to FIG. 4, according to the second embodiment.

FIG. 9 drawing which corresponds to FIG. 5, according to the second embodiment.

### DESCRIPTION OF EMBODIMENTS

Embodiments of the present invention will be described in detail hereinafter based on the drawings. The following embodiments are merely preferred examples in nature, and are not intended to limit the scope, applications, and use of the invention.

### First Embodiment of the Invention

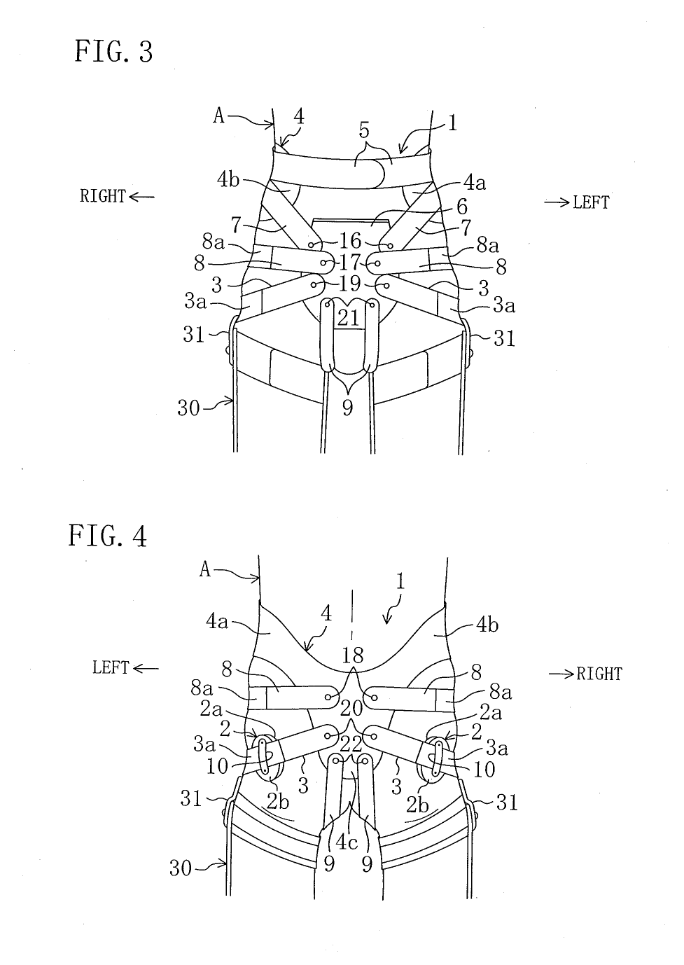

FIG. 3 to FIG. 5 show a pelvic belt 1 when used according to the first embodiment of the present invention. The pelvic belt 1 is used, for example, during rehabilitation of Patient A who has trouble walking due to such as paralysis. In the descriptions of this embodiment, the anatomy around the human pelvis and femurs will be explained before describing the structure and use instructions of the pelvic belt 1.

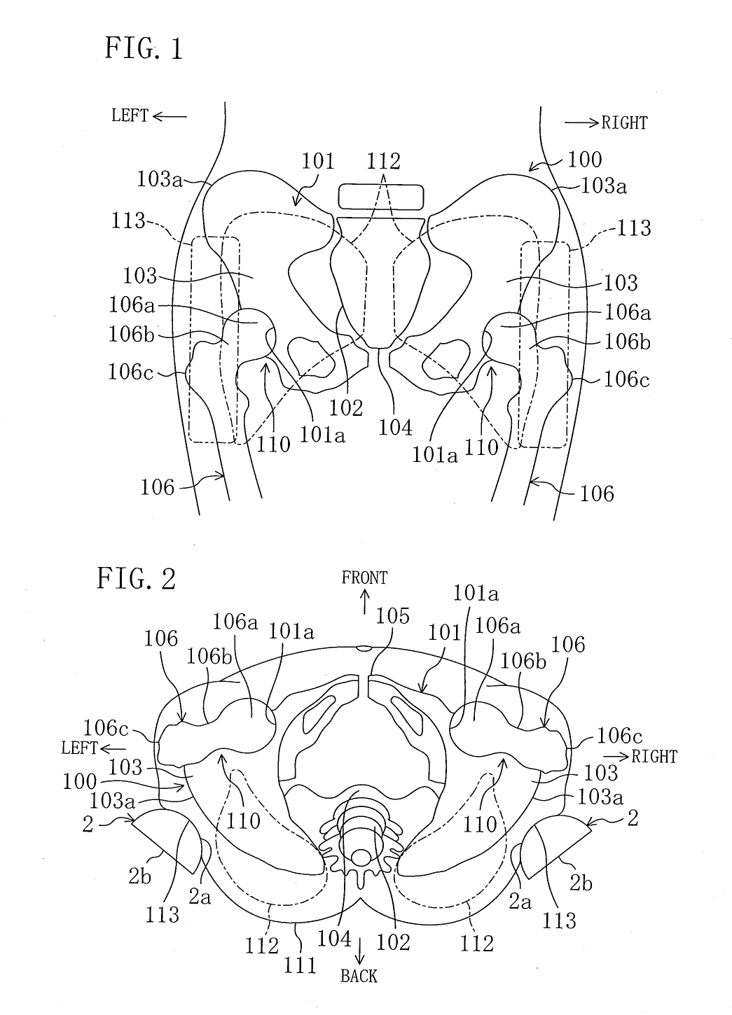

As shown in FIG. 1, the pelvis 100 includes the coxal bone 101, the sacrum 102 at the back and in the middle of the pelvis along the lateral direction, and the coccyx (not shown) at the front of the middle portion. The coxal bone 101 consists of the ilium 103, the ischium 104, and the pubis 105 (shown in FIG. 2) fused together. The ilium 103 includes the wing of ilium 103a which expands laterally.

The coxal bone 101 has the acetabulum 101a for accommodating the femur head 106a at the proximal end of the femur 106. The acetabulum 101a and the proximal end of the femur 106 form the hip joint 110. The proximal end of the femur 106 includes the femur head 106a, and also includes the femur neck 106b and the greater trochanter 106c of the femur. Further, the hips 111 (shown in FIG. 2) have plenty of muscles, such as the gluteus maximus 112, and subcutaneous tissues. No thick muscle like the gluteus maximus 112 exists in an area of the hips 111 that extends from about an outer edge of the gluteus maximus 112 in the horizontal direction toward a lateral side. Thus, a dimple 113 (indicated in phantom line in FIG. 1) called Post Trochanteric Groove (PTG) is formed. The dimple 113 is located right behind the hip joint 110. The femur head 106a, the femur neck 106b, and the greater trochanter 106c of the femur correspond to the inside of the dimple 113, and are sequentially located from the inner side to the outer side of the dimple 113 along the lateral direction.

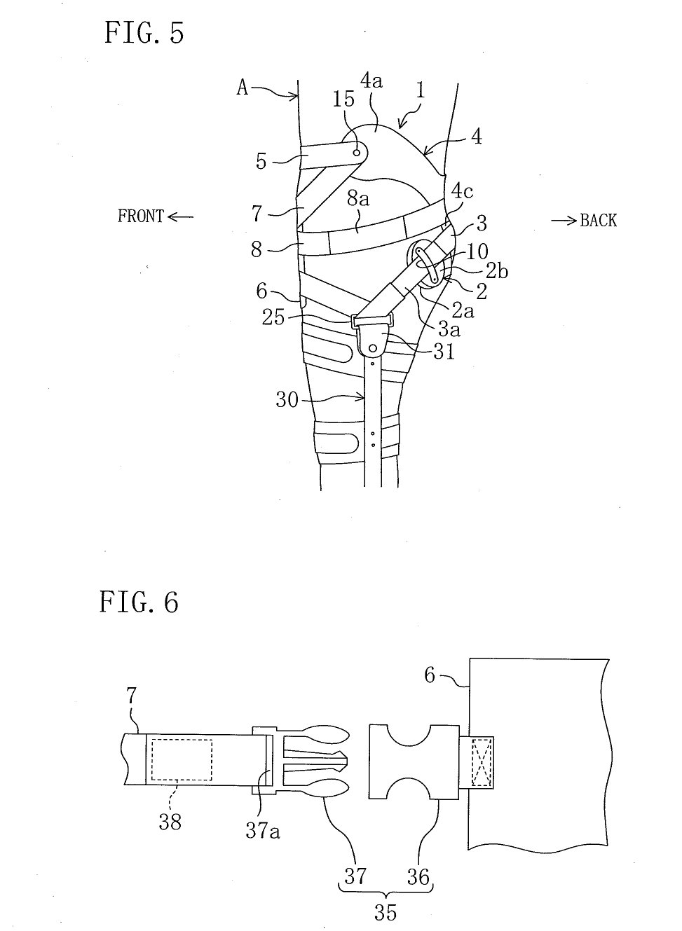

Now, a structure of the pelvic belt 1 will be described based on FIGS. 3-5. The pelvic belt 1 includes: pressing members 2, 2 (see FIG. 2) to be positioned in the left and right dimples 113 of the hips 111 that are right behind the hip joints 110; left and right pressing-member fixing bands 3, 3 for fixing the pressing members 2, 2, with the pressing members 2, 2 pushed toward the hip joints 110; a pelvis back support panel 4 configured to cover a portion corresponding to the backside of the pelvis 100; panel fixing bands 5, 5 (shown in FIG. 3 and FIG. 5) for fixing the pelvis back support panel 4; a pubic support panel 6 (shown in FIG. 3 and FIG. 5) for covering the pubic symphysis of the coxal bone 101; left and right connecting bands 7, 7 for connecting the pelvis back support panel 4 and the pubic support panel 6 together; left and right hip-joint lateral support bands 8, 8; and left and right perineum bands 9, 9 (shown in FIG. 3 and FIG. 4).

In the descriptions of the present embodiment, the word “left” refers to the left side of Patient A wearing the pelvic belt 1, and the word “right” refers to the right side of Patient A.

As shown in FIG. 2, each of the pressing members 2 has a shape of a hard tennis ball generally cut in half The surface pushed against Patient A is a curved surface 2a, which protrudes and curves hemispherically, whereas the surface opposite to the curved surface 2a is a flat surface 2b. The diameter of the pressing member 2 is set to a value between 50 mm and 100 mm, both inclusive. The hardness of the pressing member 2 on the side of the curved surface 2a is set to generally the same as the hardness of a hard tennis ball, and thus, the pressing member 2 is not much deformed when pushed toward the hip joints 110. As shown in FIG. 4 and FIG. 5, the flat surface 2b of each of the pressing members 2 has a looped insertion portion 10 for inserting the pressing-member fixing band 3. The size of the pressing member 2 can be changed according to the sex, physical size, conditions, etc. of Patient A. Similarly, the hardness and shape of the pressing member 2 can also be changed. The pressing member 2 on the side of the curved surface 2a may be made of a soft material such as a gel material. The size of the pressing member 2 may be smaller than half the size of a hard tennis ball.

As shown in FIG. 4, the pelvis back support panel 4 extends from a portion corresponding to the left wing of ilium 103a of Patient A, through a portion corresponding to the sacrum 102, to a portion corresponding to the right wing of ilium 103a to support these bones. Left and right wing-of-ilium covers 4a, 4b which extend upward and which respectively support the portions corresponding to the left and right wings of ilium 103a, 103a, are provided at both lateral sides of the pelvis back support panel 4. A sacrum cover 4c which extends downward and which supports the portion corresponding to the sacrum 102, is provided at a middle portion of the pelvis back support panel 4 along the lateral direction.

As shown in FIG. 5, one end of the left panel fixing band 5 is connected to the left wing-of-ilium cover 4a of the pelvis back support panel 4 by a rivet 15, so as to be vertically rotatable. The other end of the left panel fixing band 5 extends toward the right side. One end of the right panel fixing band 5, like the left band 5, is connected to the right wing-of-ilium cover 4b of the pelvis back support panel 4 by a rivet (not shown), so as to be vertically rotatable. The other end of the right panel fixing band 5 extends toward the left side. The left and right panel fixing bands 5, 5 overlap with each other in front of the abdomen, and are attached to each other by a hook-and-loop fastener (not shown). The tightness of the panel fixing bands 5 can be adjusted by changing the attachment position of the hook-and-loop fastener.

As shown in FIG. 3, the pubic support panel 6 is placed vertically under the panel fixing bands 5, and is apart from the panel fixing bands 5. The pubic support panel 6 extends vertically. One end of the left connecting band 7 is connected to the upper left end portion of the pubic support panel 6 by a rivet 16, so as to be vertically rotatable. The other end of the left connecting band 7 is connected to the left wing-of-ilium cover 4a by the same rivet 15 used for connecting the left panel fixing band 5, so as to be vertically rotatable. Similarly, one end of the right connecting band 7 is connected to the upper right end portion of the pubic support panel 6 by a rivet 16. The other end of the right connecting band 7 is connected to the right wing-of-ilium cover 4b by the same rivet used for connecting the right panel fixing band 5, so as to be vertically rotatable.

One end of the left hip-joint lateral support band 8 is connected to the left side of a middle portion along a vertical direction of the pubic support panel 6 by a rivet 17, so as to be vertically rotatable. As shown in FIG. 4, the other end of the left hip-joint lateral support band 8 is connected to the left side of the sacrum cover 4c of the pelvis back support panel 4 by a rivet 18, so as to be vertically rotatable. Similarly, the right hip-joint lateral support band 8 is connected to the right side of the middle portion along the vertical direction of the pubic support panel 6, and to the right side of the sacrum cover 4c of the pelvis back support panel 4. The left and right hip-joint lateral support bands 8, 8 include, at middle portions along their respective longitudinal directions, elastic portions 8a, 8a made of rubber which is elastic in the longitudinal directions.

One end of the left pressing-member fixing band 3 is connected to a lower left portion of the pubic support panel 6 by a rivet 19, so as to be vertically rotatable. This pressing-member fixing band 3 is wound around a portion corresponding to the pelvis 100. As shown in FIG. 4, the other end of the left pressing-member fixing band 3 is connected, by a rivet 20, to the sacrum cover 4c of the pelvis back support panel 4 under the rivet 18 so as to be vertically rotatable. The left pressing-member fixing band 3 includes, at a midpoint of its longitudinal dimension, an elastic portion 3a which is elastic in the longitudinal direction like the elastic portions 8a. The elastic portion 3a is inserted through the insertion portion 10 of the pressing member 2, and the elastic portion 3a is in contact with the flat surface 2b of the pressing member 2. The elasticity of the elastic portion 3a can be freely determined, thereby making it possible to adjust the pushing force that pushes the pressing member 2 toward the hip joint. The pressing-member fixing band 3 is simply inserted through the insertion portion 10 of the pressing member 2. Thus, it is possible to easily change the location of the pressing member 2 with respect to the pressing-member fixing band 3.

As shown in FIG. 5, a ring 25 is provided at a midpoint of the longitudinal dimension of the left pressing-member fixing band 3. The ring 25 is fixed to a fastening portion 31 provided at the upper end of the lower limb support 30 affixed to the left lower limb of Patient A. The lower limb support 30 is commonly used by patients having paralyzed lower limbs. Thus, detailed descriptions are not provided. The location of the fastening portion 31 can be adjusted along the vertical direction. The location of the pressing member 2 can also be changed by adjusting the location of the fastening portion 31.

The ring 25 is positioned at a location vertically lower than the pressing member 2 when affixed to Patient A. Thus, when affixed to Patient A, the pressing-member fixing band 3 extends obliquely downward from the sacrum 102 side toward the left, and reaches the fastening portion 31 of the lower limb support 30 as shown in FIG. 4, and is then bent and extends obliquely upward toward the pubis 105 as shown in FIG. 5.

Similarly, the right pressing-member fixing band 3 is connected to an lower right portion of the pubic support panel 6, and to a right side of the sacrum cover 4c of the pelvis back support panel 4. Further, an elastic portion 3a and a ring (not shown) fixed to a fastening portion 31 of the lower limb support 30 are provided at midpoints of the longitudinal dimension of the right pressing-member fixing band 3.

As shown in FIG. 3, one end of the left perineum band 9 is connected to a lower left portion of the pubic support panel 6 by a rivet 21, so as to be laterally rotatable. The left perineum band 9 passes through the left side of the Patient A's perineum, and extends toward the backside. The other end of the left perineum band 9 is connected, by a rivet 22, to the sacrum cover 4c of the pelvis back support panel 4 under the rivet 20 so as to be laterally rotatable. Similarly, the right perineum band 9 is connected to the pubic support panel 6 and the sacrum cover 4c. These perineum bands 9, 9 prevent the pelvis back support panel 4 and the pubic support panel 6 from shifting upward.

The connecting bands 7, the hip-joint lateral support bands 8, and the perineum bands 9 can be detached from the pelvis back support panel 4 and the pubic support panel 6. Further, the length of each of the connecting bands 7, the hip-joint lateral support bands 8, the pressing-member fixing bands 3, and the perineum bands 9 can be freely determined according to Patient A.

Now, use instructions of the pelvic belt 1 having the above structure will be described. First, the lower limb support 30, and then the pelvic belt 1, are affixed to Patient A. When the pelvic belt 1 is affixed, the left and right pressing members 2, 2 are positioned in the dimples 113, 113 of the hips 111. As shown in FIG. 2, each of the pressing members 2 is preferably positioned at a location at which the curved surface 2a is generally directed toward the femur head 106a to press inward from behind and obliquely behind the pelvis. The location of each pressing member 2 can be easily adjusted because the pressing-member fixing band 3 is simply inserted through the insertion portion 10 of the pressing member 2. Further, the inclination angle of the pressing-member fixing band 3 is determined so that the pressing member 2 can be stable in the dimple 113, by adjusting the location of the fastening portion 31 of the lower limb support 30. Further, the tightness of the panel fixing bands 5 is adjusted as well.

After the pelvic belt 1 is affixed to Patient A, the pressing members 2 are pushed toward the hip joints 110 by the pressing-member fixing bands 3, and the hip joints 110 are pressed from behind the pelvis. Here, the pressing members 2, 2 are located in the dimples 113, 113 in which there is no thick muscle, and therefore, the pressing force of the pressing members 2, 2 can be reliably applied to the hip joints 110. As a result, the hip joints 110 are prevented from becoming unstable and moving backward during walking, thereby' reducing the sway of the pelvis 100. Thus, it is possible to make the walking posture of Patient A more similar to a normal walking posture without applying an assisting force given by the drive unit as in the conventional case.

Further, the pressing members 2, 2 are located in the dimples 113, 113 during walking. Therefore, displacement of the pressing members 2, 2 does not tend to occur, and it is possible to stabilize the pressing members 2, 2.

Because such a conventional drive unit is not necessary, the fixing bands 3 may be tightened just enough to bring the pressing members 2 pressed against the hip joints 110. Thus, the movement of the pelvis 100 in a normal range is not interrupted by the pelvic belt 1, and it is possible to fine adjust the location at which the pressing-member fixing bands 3 are wound, and the locations of the pressing members 2, according to the paralytic condition and degree of restoration of each Patient A.

Further, in the first embodiment, the pelvis back support panel 4 and the pubic support panel 6 can support the pelvis 100 of Patient A from the backside and the front side. Thus, it is possible to make the walking postures of the patients with severe conditions, as well, more similar to a normal walking posture.

As described above, according to the pelvic belt 1 of the first embodiment, the pressing members 2, 2 positioned at portions corresponding to the backside of the hip joints 110 of Patient A are pushed against the hip joints 110 and fixed, by the pressing-member fixing bands 3 wound around a portion corresponding to the pelvis 100. Thus, it is possible to make the walking posture of Patient A more similar to a normal walking posture without using the conventional drive unit. This structure allows the pelvis 100 to move in a normal range, thereby making it possible to have a sufficient facilitating effect on a muscle. Also, it is possible to obtain a sufficient effect of treatment by adjusting the locations of the pressing members 2, 2 according to the paralytic condition and degree of restoration of each Patient A. Moreover, the structure can be simplified, and the cost can be reduced.

Further, the surface of each pressing member 2 that is pushed toward the hip joint 110 is the curved surface 2a. Thus, the pressing force of the pressing member 2 can be reliably applied to the hip joint 110, without causing pain to Patient A when the pressing member 2 is affixed.

Here, in the first embodiment, the pressing members 2, 2 are pushed against both lateral sides of Patient A. However, the structure is not limited to this structure, and the pressing member 2 may be pressed against only one side.

Further, the panel fixing bands 5, the connecting bands 7, and the perineum bands 9 may include elastic portions like the elastic portions 3a of the pressing-member fixing bands 3.

Further, the connecting bands 7 and the perineum bands 9 may be omitted. The pelvis back support panel 4 and the pubic support panel 6 may be omitted.

Further, in the first embodiment, the connecting bands 7 are fixed to the pubic support panel 6 by the rivets 16. However, the structure is not limited to this structure. For example, as in a variation shown in FIG. 6, a one-touch connector 35 may be used to connect the connecting bands 7 to the pubic support panel 6. The connector 35 includes a female member 36 attached to the pubic support panel 6, and a male member 37 attached to the connecting band 7. The male member 37 is disposed in the female member 36, and is locked in this state so as not to be disengaged from the female member 36. Further, the end of the connecting band 7 is inserted through a loop portion 37a of the male member 37, and is fixed around a middle portion of the connecting band 7 along its longitudinal direction by a hook-and-loop fastener 38. The length of the connecting band 7 can be changed according to patients by changing where to fix the hook-and-loop fastener 38. In the case where the connecting band 7 is connected to the pelvis back support panel 4, as well, the connector 35 and the hook-and-loop fastener 38 can be used.

Although not shown in the drawings, the connector and the hook-and-loop fastener according to the variation can be used not only for the connection in the above example, but also for the connection of the pressing-member fixing bands 3, the panel fixing bands 5, the hip-joint lateral support bands 8, and the perineum bands 9 to the pubic support panel 6 and the pelvis back support panel 4.

### Second Embodiment of the Invention

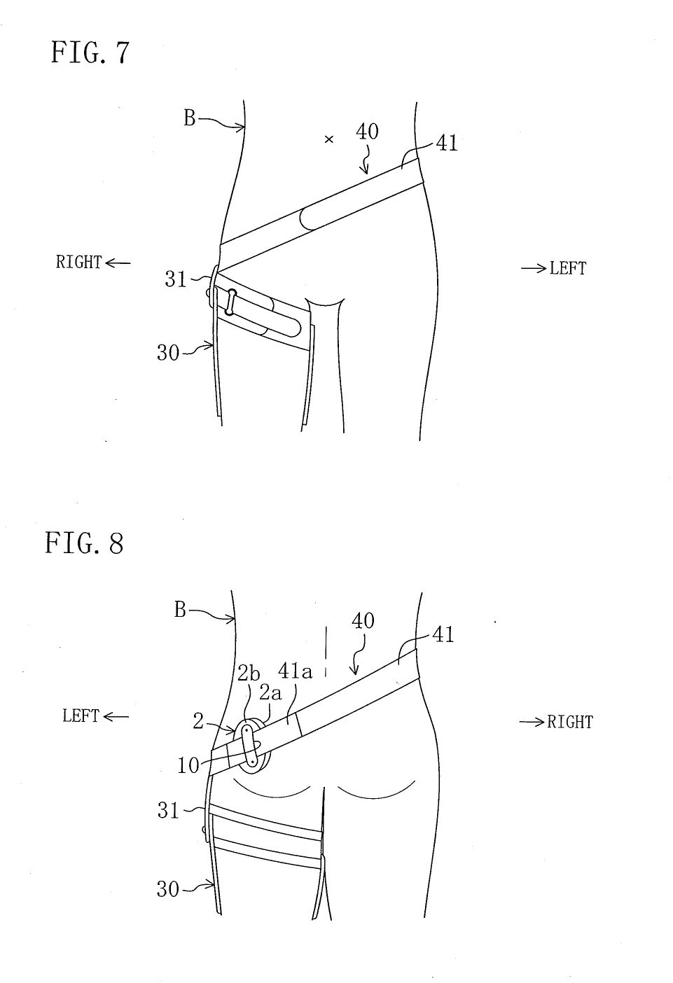

FIG. 7 to FIG. 9 show a pelvic belt 40 when used according to the second embodiment of the present invention. The pelvic belt 40 of the second embodiment differs from the pelvic belt 1 of the first embodiment in that the pelvic belt 40 does not include the pelvis back support panel 4, the panel fixing bands 5, the pubic support panel 6, the connecting bands 7, the hip-joint lateral support bands 8, and the perineum bands 9. The pelvic belt 40 is used for Patient B in better conditions than Patient A who uses the pelvic belt 1 of the first embodiment.

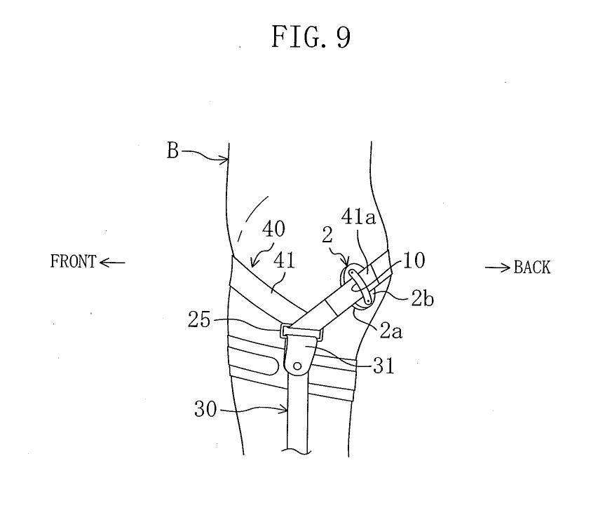

Specifically, as shown in FIG. 8 and FIG. 9, the pelvic belt 40 of the second embodiment includes one pressing member 2 and a pressing-member fixing band 41, and is suitable for rehabilitation of Patient B whose one lower limb is paralyzed. In the second embodiment, the case in which the pelvic belt 40 is used for Patient B whose left lower limb is paralyzed is described.

The pressing-member fixing band 41 forms a loop wound around a portion corresponding to the pelvis 100, and has an elastic portion 41a in its middle portion. The elastic portion 41a is inserted through an insertion portion 10 of the pressing member 2. . When affixed to Patient B, the left side of the pressing-member fixing band 41 is located at the lowermost position, and the pressing-member fixing band 41 extends obliquely upward from the lowermost position toward the right side. As shown in FIG. 9, a ring 25 is provided on the left side of the pressing-member fixing band 41. Further, the right side of the pressing-member fixing band 41 is located at a portion corresponding to an upper portion of the wing of ilium 103a.

According to the pelvic belt 40 of the second embodiment, as well, the pressing member 2 positioned at a portion corresponding to the backside of the hip joint 110 of Patient B is pushed against the hip joint 110 and fixed, by the pressing-member fixing band 41 wound around a portion corresponding to the pelvis 100. Thus, the same effects as in the first embodiment can be obtained.

Although not shown in the drawings, it is possible to use the pelvic belt 40 of the second embodiment for those patients whose right lower limb is paralyzed. Further, two pelvic belts 40 can be used to push two pressing members 2 against both hip joints.

Further, the lower limb support 30 used together with the pelvic belts 1, 40 of the first and second embodiments may have various shapes and structures. Specifically, although not shown in the drawings, a band-like support for winding a lower limb (a femur) and provided with a ring can be used together with the pelvic belt 1, 40 so that the pressing-member fixing band 4, 41 can be inserted through the ring.

Further, the length of the pressing-member fixing band 41 of the second embodiment may be adjusted by providing a one-touch connector and a hook-and-loop fastener as in the variation of the first embodiment.

### INDUSTRIAL APPLICABILITY

As explained above, the pelvic belt according to the present invention can be used, for example, for rehabilitation of patients who have trouble walking due to such as paralysis.

### DESCRIPTION OF REFERENCE CHARACTERS

1, 40 pelvic belt

2 pressing member

2a curved surface

3 pressing-member fixing band (fixing member)

3a elastic portion

4 pelvis back support panel (pelvis back support)

6 pubic support panel (pubic support)

10 insertion portion

100 pelvis

101 coxal bone

102 sacrum

103a wing of ilium

110 hip joint

111 hip

112 gluteus maximus

113 dimple

1. An equipment to be put on a lower body, comprising:
a pressing member for pressing a hip joint; and
a fixing member for pushing the pressing member from a backside toward the hip joint and fixing the pressing member, wherein
a pressing surface of the pressing member is curved and protrudes, and
the fixing member is configured to fix the pressing member to a dimple of the hips, the dimple extending from about an outer edge of the gluteus maximus along a horizontal direction toward a lateral side.

2. The equipment to be put on the lower body of claim 1, wherein
the fixing member is a pressing-member fixing band, and the pressing member includes an insertion portion through which the pressing-member fixing band is inserted.

3-4. (canceled)

5. The equipment to be put on the lower body of claim 1, wherein the fixing member has an elongated shape, and has an elastic portion that is elastic in a longitudinal direction of the fixing member.

6. The equipment to be put on the lower body of claim 1, comprising a pelvis back support for covering a portion corresponding to the wing of ilium of the coxal bone and the sacrum.

7. The equipment to be put on the lower body of claim 1, comprising a pubic support for covering a portion corresponding to the pubic symphysis of the coxal bone.

8. The equipment to be put on the lower body of claim 1, wherein
the fixing member is a pressing-member fixing band,
the equipment includes:
a pelvis back support panel to which one end of the pressing-member fixing band is connected and which covers a backside of the pelvis;
a pubic support panel to which the other end of the pressing-member fixing band is connected and which covers the pubic symphysis of the coxal bone; and
a hip joint lateral support band which is connected to the pelvis back support panel and the pubic support panel and which extends over a lateral side of the hip joint, and
the pressing member is fixed, by the pressing-member fixing band, to a dimple of the hips that is formed behind the hip joint and between an outer edge of the gluteus maximus in a horizontal direction and the greater trochanter of the femur, so as to press the hip joint from behind, thereby preventing the hip joint from becoming unstable and moving backward.

9. The equipment to be put on the lower body of claim 6, comprising at least one of:
a panel fixing band which is connected to the pelvis back support panel and wound around the abdomen, for fixing the pelvis back support panel to a position at which the pelvis back support panel covers the backside of the pelvis; and
a connecting band which is connected to the pelvis back support panel and the pubic support panel, and which extends over a lateral side of the abdomen.

10. The equipment to be put on the lower body of claim 1, wherein
the fixing member is a pressing-member fixing band, the pressing-member fixing band forms a loop shape for being wound around a portion corresponding to the pelvis, and fixes the pressing member to one of left and right dimples in the hips, each of the dimples extending from about an outer edge of the gluteus maximus along a horizontal direction toward a lateral side, so as to press the hip joint from behind, thereby preventing the hip joint from becoming unstable and moving backward, and
a portion of the pressing-member fixing band that is opposite, along a lateral direction of the pressing-member fixing band, to a portion at which the pressing member is fixed is wound around a portion corresponding to an upper portion of the wing of ilium.
